(19) United States
(12) Patent Application Publication
(10) Pub. No.: US 2019/0091438 A1
(43) Pub. Date: Mar. 28, 2019

(54) CATHETER WITH SIDE PORTS AND METHODS OF USE

(71) Applicant: Cardiovascular Systems, Inc., St. Paul, MN (US)

(72) Inventors: Joseph P. Higgins, Minnetonka, MN (US); William G. Besser, Minneapolis, MN (US)

(21) Appl. No.: 16/139,872

(22) Filed: Sep. 24, 2018

Related U.S. Application Data
(60) Provisional application No. 62/563,241, filed on Sep. 26, 2017.

(51) Int. Cl. A61M 25/00; A61M 25/01; A61M 25/10

(52) U.S. Cl. CPC A61M 25/007; A61M 25/0023; A61M 25/0043; A61M 25/10; A61M 2025/0073; A61M 25/0105

(57) ABSTRACT

A catheter with a lumen and comprising side port(s) through the elongate body of the catheter at predetermined locations. The side port(s) may be positioned at anatomical locations of interest and a fluid, e.g., a medicament, infused down the catheter's lumen, the fluid flowing out of the side port(s) to treat the anatomical location. Alternative embodiments may include a translatable inner sheath that may cover some of the side port(s) and may also include an aperture that may align with at least one of the side port(s). The side ports may be opened and closed by means of an acuator such as a push/pull wire or an electrically response shape memory material. A selective filter may be used to cover the side ports, whereby only fluids of certain viscosities and/or molecular size may pass through the filter and side port.

### CROSS-REFERENCE TO RELATED APPLICATIONS

This application claims priority to App. Ser. No. 62/563,241, entitled, CATHETER WITH SIDE PORTS AND METHOD OF USE, filed Sep. 26, 2017, the entire contents of which are incorporated by reference.

### STATEMENT REGARDING FEDERALLY SPONSORED RESEARCH OR DEVELOPMENT

Not Applicable

### FIELD OF THE INVENTION

The invention relates generally to catheters used generally in combination with medical devices requiring intraluminal access, more specifically in combination with devices and systems for accessing and navigating through blood vessels, facilitating positioning of devices in blood vessels and delivering fluids to the blood vessels.

### DESCRIPTION OF THE RELATED ART

It is common to position catheters to aid in the diagnosis and treatment of various medical disorders such as treating occlusions, lesions or stenotic material in a blood vessel using, e.g., and without limitation, atherectomy devices and systems and angioplasty systems.

During these exemplary intraluminal procedures, it is common to introduce fluids down the catheter lumen for release at the distal end of the catheter, with some catheters comprising ports or holes through the catheter wall near the distal end of the catheter. However, it would be advantageous to have the ability to target delivery of a fluid, e.g., a medicament, to specific locations within the subject lumen, from a predetermined point or points along the length of the catheter that are not near the distal end of the catheter. It would also be advantageous to control the delivery of the fluid to the specific location(s).

The present invention overcomes these deficiencies and provides, inter alia, the above-referenced improvements.

### BRIEF SUMMARY OF THE INVENTION

The present system is directed in various methods, devices and systems relating to a catheter with a lumen and comprising side port(s) through the elongate body of the catheter at predetermined locations. The side port(s) may be positioned at anatomical locations of interest and a fluid, e.g., a medicament, infused down the catheter's lumen, the fluid flowing out of the side port(s) to treat the anatomical location. Alternative embodiments may include a translatable inner sheath that may cover some of the side port(s) and may also include an aperture that may align with at least one of the side port(s). The side ports may be opened and closed by means of an acuator such as a push/pull wire or an electrically response shape memory material. A selective filter may be used to cover the side ports, whereby only fluids of certain viscosities and/or molecular size may pass through the filter and side port. The side ports may be arranged in an at least semi-annular pattern and adjacent side ports may be spaced apart longitudinally and/or circumferentially from each other.

### BRIEF DESCRIPTION OF THE DRAWINGS

FIG. 1 is a cross-sectional and cutaway view of one embodiment of the present invention;

FIG. 2 is a cross-sectional and cutaway view of one embodiment of the present invention;

FIG. 3 is a cross-sectional and cutaway view of one embodiment of the present invention;

FIG. 4A is a cross-sectional and cutaway view of one embodiment of the present invention;

FIG. 4B is a side view cutaway view of one embodiment of the present invention;

FIG. 5 is a cross-sectional and cutaway view of one embodiment of the present invention;

FIG. 6 is a cross-sectional and cutaway view of one embodiment of the present invention;

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

For the purposes of the present invention, the following terms and definitions apply:

“Bodily disorder” refers to any condition that adversely affects the function of the body.

The term “treatment” includes prevention, reduction, delay, stabilization, and/or elimination of a bodily disorder, e.g., a vascular disorder. In certain embodiments, treatment comprises repairing damage cause by the bodily, e.g., vascular, disorder and/or intervention of same, including but not limited to mechanical intervention.

A “therapeutic agent” comprises any substance capable of exerting an effect including, but not limited to therapeutic, prophylactic or diagnostic. Thus, therapeutic agents may comprise anti-inflammatories, anti-infectives, analgesics, anti-proliferatives and the like including but not limited to antirestenosis drugs and may further include anti-vasospasm drugs. Therapeutic agent further comprises mammalian stem cells. Therapeutic agent as used herein further includes other drugs, genetic materials and biological materials. The genetic materials mean DNA or RNA, including, without limitation, of DNA/RNA encoding a useful protein, intended to be inserted into a human body including viral vectors and non-viral vectors. Viral vectors include adenoviruses, gutted adenoviruses, adeno-associated virus, retroviruses, alpha virus, lentiviruses, herpes simplex virus, ex vivo modified cells (e.g., stem cells, fibroblasts, myoblasts, satellite cells, pericytes, cardiomyocytes, skeletal myocytes, macrophage), replication competent viruses, and hybrid vectors. Non-viral vectors include artificial chromosomes and mini-chromosomes, plasmid DNA vectors, cationic polymers, graft copolymers, neutral polymers PVP, SP1017, lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor .alpha. and .beta., platelet derived endothelial growth factor, platelet derived growth factor, tumor necrosis factor .alpha., hepatocyte growth factor and insulin like growth factor), transcription factors, proteinkinases, CD inhibitors, thymidine kinase, and bone morphogenic proteins. These dimeric proteins can be provided as homodimers, heterodimers, or combinations thereof, alone or together with other molecules.

Therapeutic agents further includes cells that can be of human origin (autologous or allogeneic) or from an animal source (xenogeneic), genetically engineered, if desired, to deliver proteins of interest at the transplant site. Cells within the definition of therapeutic agents herein further include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Therapeutic agent also includes non-genetic substances, such as: anti-thrombogenic agents such as heparin, heparin derivatives, and urokinase; anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as heparin, antithrombin compounds, platelet receptor antagonists, anti-thrombin anticodies, anti-platelet receptor antibodies, aspirin, dipyridamole, protamine, hirudin, prostaglandin inhibitors, platelet inhibitors and tick antiplatelet peptides; vascular cell growth promotors such as growth factors, Vascular Endothelial Growth Factors, growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme, inhibitors including captopril and enalopril. The biologically active material can be used with (a) biologically non-active material(s) including a solvent, a carrier or an excipient, such as sucrose acetate isobutyrate, ethanol, n-methyl pymolidone, dimethyl sulfoxide, benzyl benxoate and benzyl acetate.

Further, “therapeutic agent” includes, in particular in a preferred therapeutic method of the present invention comprising the administration of at least one therapeutic agent to a procedurally traumatized, e.g., by intraluminal access, to prevent, treat and/or inhibit vasospasm, particularly but not limited to a procedure using transradial access wherein the vasospasm may be catheter-induced. Anti-vasospasm therapeutic agents may include, but are certainly not limited to: vaso-relaxants and/or vaso-dilators, e.g., verapamil either alone or in combination with nitroglycerine, nitroglycerine, nicorandil, isosorbide mononitrate, nitrates, and nicardipine.

An alternate preferred therapeutic method comprises administration of the “therapeutic agent(s)” to a procedurally traumatized, e.g., by an angioplasty or atherectomy procedure, mammalian vessel to inhibit restenosis. Preferably, the therapeutic agent is a cytoskeletal inhibitor or a smooth muscle inhibitor, including, for example, taxol and functional analogs, equivalents or derivatives thereof such as taxotere, paclitaxel, abraxane TM, coroxane TM or a cytochalasin, such as cytochalasin B, cytochalasin C, cytochalasin A, cytochalasin D, or analogs or derivatives thereof.

Additional specific examples of “therapeutic agents” that may be applied to a bodily lumen using various embodiments of the present invention comprise, without limitation: L-Arginine; Adipose Cells; Genetically altered cells, e.g., seeding of autologous endothelial cells transfected with the beta-galactosidase gene upon an injured arterial surface; Erythromycin; Penicillin: Heparin; Aspirin; Hydrocortisone; Dexamethasone; Forskolin; GP IIb-IIIa inhibitors; Cyclohexane; Rho Kinsase Inhibitors; Rapamycin; Histamine; Nitroglycerin; Vitamin E; Vitamin C; Stem Cells; Growth Hormones; Hirudin; Hirulog; Argatroban; Vapirprost; Prostacyclin; Dextran; Erythropoietin; Endothelial Growth Factor; Epidermal Growth Factor; Core Binding Factor A; Vascular Endothelial Growth Factor; Fibroblast Growth Factors; Thrombin; Thrombin inhibitor; and Glucosamine, among many other therapeutic substances.

The therapeutic agent delivery system of the present invention can be used to apply the therapeutic agent to any wall surface of a biological lumen where a catheter can be inserted. Such biological lumen includes, inter alia, blood vessels, urinary tract, coronary vasculature, esophagus, trachea, colon, and biliary tract.

A therapeutically effective, or therapeutic, or effective, dose refers to that amount of therapeutic agent, which mitigates and/or provides therapy for the symptoms or condition. As the skilled artisan will readily recognize, therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. Pharmaceutical formulations which exhibit large therapeutic indices are preferred. The data obtained from cell culture assays and animal studies are used to formulate a range of dosage for human use. The dosage contained in such formulations is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy.

Generally, the present invention comprises various embodiments that provide ports through the side wall of the catheter, so that fluid moving down the lumen of the catheter may be expelled from the port(s) at a desired location within the bodily lumen. Thus, the port(s) provide a fluid communication channel between the catheter lumen and the environment located outside of the catheter, e.g., a blood vessel wall. In some embodiments, the port(s) may comprise structure that enables the port(s) to selectively open and/or close. Other embodiments may comprise structure that enables molecules of a certain maximum size to pass through the port(s) while excluding other molecules larger than the maximum size. Still other embodiments may comprise structure that enables fluid of a predetermined viscosity to pass through the port(s) while excluding other fluids.

In certain embodiments, the fluid infused through the catheter and port(s) may comprise saline while in other embodiments the fluid may comprise one or more therapeutic agents, e.g., for treating and/or preventing of restenosis, therapeutic cells including but not limited to stem cells

The various embodiments of the present invention are illustrated in the Figures, generally comprising a catheter with at least one side port or access channel through the side wall that may be opened and/or closed. Further, the opening and/or closing may be complete or may be partial, so that the size of the at least one side port or access channel may be modified, and therefore the amount of fluid flowing therethrough to a target region of the body, e.g., a lumen wall, may also be controlled.

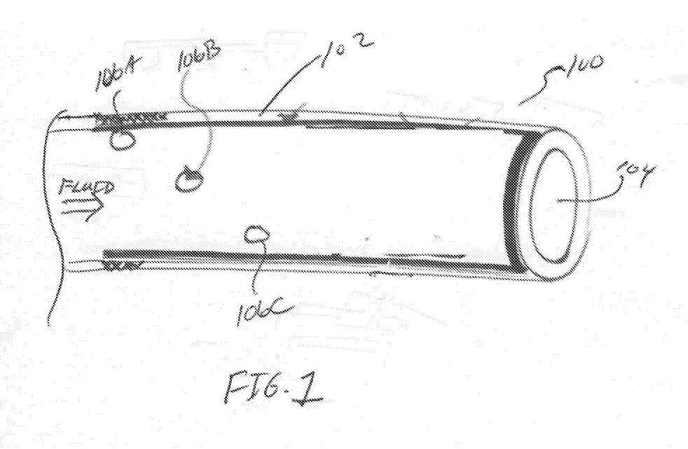

FIG. 1 provides a catheter 100 comprising an elongate body 102 and a central lumen 104 defined therethrough. A group or pattern of a plurality of side ports 106A, 106B, 106C or access channels are defined through the elongate body 102. The size, shape and distribution of the side ports or access channels 106A, 106B, 106C may be varied to control the amount of influsate fluid permitted or enabled to flow therethrough. The catheter 100 may be positioned so that one or more of the plurality of side ports 106A, 106B, 106C may be adjacent an anatomical location of interest for treating and/or preventing a vascular condition such as, without limitation, vasospasm or restenosis with an appropriate therapeutic agent(s) infused through the lumen 104 and out of at least one of the side ports 106A, 106B, 106C.

The location and pattern of the side ports 106A, 106B, 106C as illustrated are purely exemplary and may comprise any pattern. The side ports 106A, 106B, 106C may be spaced apart longitudinally from adjacent side ports and/or spaced apart circumferentially from adjacent side ports as shown in FIG. 1. Moreover, the exemplary grouping of side ports 106A, 106B, 106C of FIG. 1 may be the only grouping on a catheter 100 or there may be additional groupings of side ports that may, or may not be, separated longitudinally and/or circumferentially from an adjacent grouping of side ports.

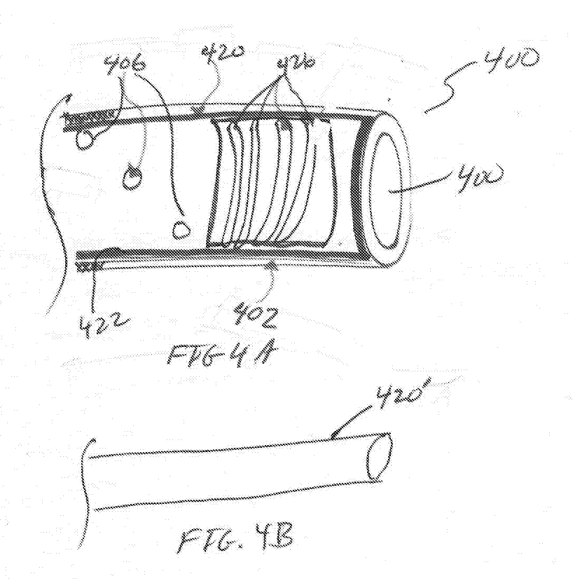

An inner sheath (see also FIG. 4) with, or without side apertures may be translatably and/or rotatably received within the catheter lumen 104 to selectively close (block) or open (unblock) one or more of the side ports or access channels 106A 106B, 106C, including partial opening and/or closing. As discussed above, one or more groupings of side ports 106A, 106B, 106C may be provided along the length of the catheter's elongate body 102.

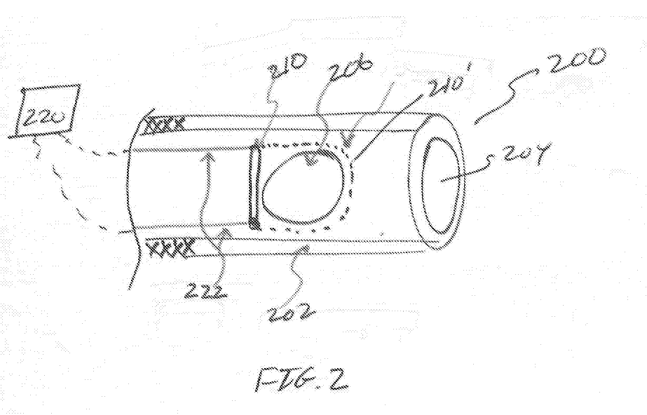

FIG. 2 illustrates a covering on the exterior surface of a catheter 200 comprising an elongate body 202 with a central lumen 204 and at least one side port 206 (or a plurality of side ports or groupings of patterns of side ports as discussed in connection with FIG. 1).

The side port 206 may be opened or actuated by, e.g., heat application from application of a current or other heat source 220 via wires 222, so that a flap 210, or similar structure, normally covering the side port 206 is activated, causing the flap to deform, e.g., unroll or otherwise expose the side port 206 located underneath the covering flap 210. The undeformed flap is shown in dashed lines at 210′, with the deformed flap 210 shown in exemplary rolled-up form at 210. Removal of the activating energy source 220 enables the deformed flap 210 to return to its undeformed 210′ covering profile. Flap 210 may comprise a shape memory material or other material responsive to deformation by application of electrical energy and/or heat energy.

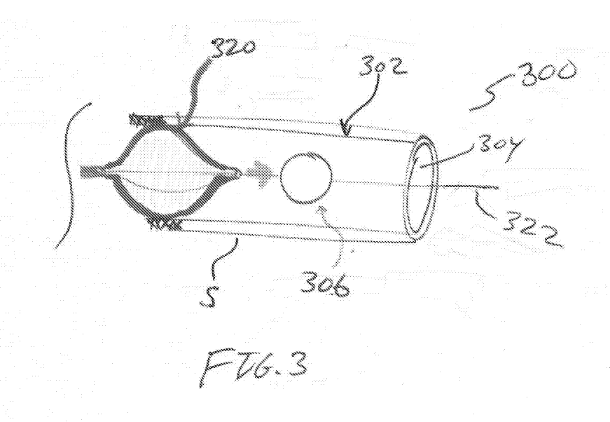

FIG. 3 shows another embodiment of a system comprising a catheter 300 defined by an elongate body 302 with a central lumen 302 therethrough and at least one, or a plurality of, side ports 306 through the elongate body 302. Infusate may flow distally through the lumen 304 and out of one or more of the side port(s) 306. A balloon catheter 320 or covered stent translatably disposed along a guide wire 322 within the catheter lumen 304, or alternatively disposed over the exterior surface S of the catheter 300, to selectively close one or more of the side ports 306 along the length of the catheter 300. The balloon 320 or covered stent may be inflated at a point distal to the selected side port(s) 306 for infusion flow to the anatomical location. In this arrangement balloon 320 will function to block the infusate from exiting at the distal end of the catheter, thereby concentrating the infusate at the designated side port(s) 306 for flow therethrough.

FIGS. 4A and 4B illustrate a system comprising a catheter 400 comprising an elongate body 402 and a central lumen 404 therethrough and with one or more (or a plurality of) side ports 406 as discussed above through the elongate body 402. An inner sheath 420 is provided that is translatably and/or rotatably received within the lumen 404 of the catheter 400 for selective opening and/or closing of the ports and/or access channels, including partial opening and/or closing.

FIG. 4A shows the inner sheath 420 with a series of apertures 426 through the elongate body 422 of the inner sheath. There may be one aperture 426 or a plurality of apertures 426. Thus, the inner sheath 420 may be translated and/or rotated within lumen 404 of catheter 400 to align the one or more apertures 426 of the inner sheath with at least one of the side ports 406 of the catheter 400. FIG. 4B provides an alternative embodiment wherein the inner sheath 420′ does not comprise any apertures. In this case, the inner sheath may be translated to cover or uncover one or more side ports 406 to enable targeted fluid flow therethrough.

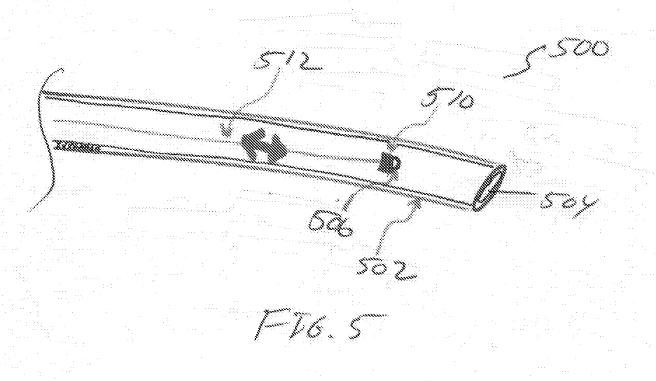

FIG. 5 illustrates an alternate embodiment of the catheter 500 comprising an elongate body 402 defining a central lumen 504 and with side port(s) 506 defined therethrough that are either biased in a closed configuration or in an open configuration. Connected with, or proximate to, an outer edge of the side port(s) 506 are structures 510, e.g., a flap structure, disposed on the inner wall, or embedded in the wall of the elongate body 502, that may be opened or closed using, e.g., a push-pull wire system 512 as shown. The flap structure 510 would preferably comprise a complementary size and shape to match the relevant side port 506 size and shape, or be slightly larger than the side port 506 hole to ensure full coverage and closure in the closed configuration.

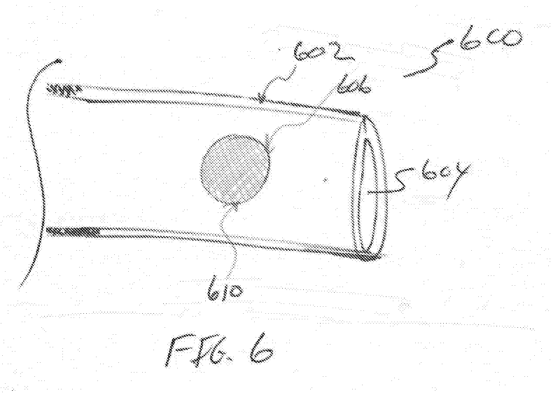

FIG. 6 illustrates another catheter 600 comprising an elongate body 602 defining a central lumen 604 with at least one side port (or a plurality thereof) 606 defined through the elongate body 602. A filter or graft or elastomer micro seal 610 is shown covering side port 606 and is adapted to allow selective fluid flow through the side port 606 that is covered by the filter, graft or elastomer micro seal 610. In this embodiment, selected fluids may be transferred through the side port 606, while others are blocked from passage therethrough, e.g., certain viscosities of fluids may be allowed, while others are excluded. In addition, molecules of a certain maximum size may be allowed through the side port 606, while others larger than the allowed maximum size are blocked from egress out of the catheter lumen by the covering structure 610.

Additional alternate embodiments include structures covering the port(s) or access channel(s) and that are biased to close, but that can be opened by the force of fluid flowing through the catheter lumen. Moreover, a one-way valve, check valve, or other valve may be provided within the port or access channel and designed to allow fluid to flow out of the valved port or access channel. In addition, the valved port or access channel may be designed to allow only fluid with a selected flow force to pass through the valve.

Related to the embodiments of FIGS. 1 and 4, an inner sheath may be provided that is selectively collapsible in order to open ports along the catheter and to provide a separate lumen that is capable of delivering fluid to the newly opened ports.

Moreover, we provide disclosure of the following patents and applications, each of which are assigned to Cardiovascular Systems, Inc., and incorporated herein in their entirety, each of which may comprise systems, methods and/or devices that may be used with various embodiments of the presently disclosed subject matter:

U.S. Pat. No. 9,468,457, “ATHERECTOMY DEVICE WITH ECCENTRIC CROWN”;

U.S. Pat. No. 9,439,674, “ROTATIONAL ATHERECTOMY DEVICE WITH EXCHANGEABLE DRIVE SHAFT AND MESHING GEARS”;

U.S. Pat. No. 9,220,529, “ROTATIONAL ATHERECTOMY DEVICE WITH ELECTRIC MOTOR”;

U.S. Pat. No. 9,119,661, “ROTATIONAL ATHERECTOMY DEVICE WITH ELECTRIC MOTOR”;

U.S. Pat. No. 9,119,660, “ROTATIONAL ATHERECTOMY DEVICE WITH ELECTRIC MOTOR”;

U.S. Pat. No. 9,078,692, “ROTATIONAL ATHERECTOMY SYSTEM”;

U.S. Pat. No. 6,295,712, “ROTATIONAL ATHERECTOMY DEVICE”;

U.S. Pat. No. 6,494,890, “ECCENTRIC ROTATIONAL ATHERECTOMY DEVICE”;

U.S. Pat. No. 6,132,444, “ECCENTRIC DRIVE SHAFT FOR ATHERECTOMY DEVICE AND METHOD FOR MANUFACTURE”;

U.S. Pat. No. 6,638,288, “ECCENTRIC DRIVE SHAFT FOR ATHERECTOMY DEVICE AND METHOD FOR MANUFACTURE”;

U.S. Pat. No. 5,314,438, “ABRASIVE DRIVE SHAFT DEVICE FOR ROTATIONAL ATHERECTOMY”;

U.S. Pat. No. 6,217,595, “ROTATIONAL ATHERECTOMY DEVICE”;

U.S. Pat. No. 5,554,163, “ATHERECTOMY DEVICE”;

U.S. Pat. No. 7,507,245, “ROTATIONAL ANGIOPLASTY DEVICE WITH ABRASIVE CROWN”;

U.S. Pat. No. 6,129,734, “ROTATIONAL ATHERECTOMY DEVICE WITH RADIALLY EXPANDABLE PRIME MOVER COUPLING”;

U.S. patent application Ser. No. 11/761,128, “ECCENTRIC ABRADING HEAD FOR HIGH-SPEED ROTATIONAL ATHERECTOMY DEVICES”;

U.S. patent application Ser. No. 11/767,725, “SYSTEM, APPARATUS AND METHOD FOR OPENING AN OCCLUDED LESION”;

U.S. patent application Ser. No. 12/130,083, “ECCENTRIC ABRADING ELEMENT FOR HIGH-SPEED ROTATIONAL ATHERECTOMY DEVICES”;

U.S. patent application Ser. No. 12/363,914, “MULTI-MATERIAL ABRADING HEAD FOR ATHERECTOMY DEVICES HAVING LATERALLY DISPLACED CENTER OF MASS”;

U.S. patent application Ser. No. 12/578,222, “ROTATIONAL ATHERECTOMY DEVICE WITH PRE-CURVED DRIVE SHAFT”;

U.S. patent application Ser. No. 12/130,024, “ECCENTRIC ABRADING AND CUTTING HEAD FOR HIGH-SPEED ROTATIONAL ATHERECTOMY DEVICES”;

U.S. patent application Ser. No. 12/580,590, “ECCENTRIC ABRADING AND CUTTING HEAD FOR HIGH-SPEED ROTATIONAL ATHERECTOMY DEVICES”;

U.S. patent application Ser. No. 29/298,320, “ROTATIONAL ATHERECTOMY ABRASIVE CROWN”;

U.S. patent application Ser. No. 29/297,122, “ROTATIONAL ATHERECTOMY ABRASIVE CROWN”;

U.S. patent application Ser. No. 12/466,130, “BIDIRECTIONAL EXPANDABLE HEAD FOR ROTATIONAL ATHERECTOMY DEVICE”; and

U.S. patent application Ser. No. 12/388,703, “ROTATIONAL ATHERECTOMY SEGMENTED ABRADING HEAD AND METHOD TO IMPROVE ABRADING EFFICIENCY”

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A system comprising:
a catheter comprising
an elongate body and a central lumen,
a plurality of side ports defined through the elongate body,
wherein the plurality of side ports are arranged in at least one grouped pattern that is at least semi-annular.

2. The system of claim 1, wherein a plurality of grouped patterns of side ports are disposed along at least a portion of the elongated body of the catheter, each grouped pattern in the plurality of side ports spaced longitudinally and/or circumferentially apart from an adjacent grouped pattern of side ports.

3. The system of claim 1, wherein at least some of the plurality of side ports are spaced apart longitudinally and/or circumferentially from adjacent side ports.

4. The system of claim 1, wherein the side ports are of varying size.

5. The system of claim 2, wherein the side ports within each grouped pattern of side ports are of varying size.

6. The system of claim 1, further comprising a fluid infused through the central lumen of the catheter.

7. The system of claim 1, further comprising an inflatable balloon translatably disposed within the central lumen of the catheter.

8. The system of claim 1, further comprising an inner sheath comprising an elongate body and a central lumen therethrough, the inner sheath adapted to be rotatable and translatable within the central lumen of the catheter and to selectively cover or uncover one or more of the plurality of side ports.

9. The system of claim 8, wherein the inner sheath further comprises at least one aperture through the elongate body of the inner sheath, the inner sheath configured to align the at least one aperture of the inner sheath with at least one side port.

10. The system of claim 9, further comprising a fluid infused through the central lumen of the inner sheath.

11. The system of claim 10, further comprising an inflatable balloon translatably disposed within the central lumen of the inner sheath.

12. A system comprising:
a catheter comprising
an elongate body and a central lumen, configured to allow an infusion fluid to flow therethrough,
at least one side port defined through the elongate body, configured to allow the infusion fluid to flow therethrough.

13. The system of claim 12, further comprising an inflatable balloon translatably disposed within the central lumen of the catheter.

14. The system of claim 12, further comprising an inner sheath comprising an elongate body and a central lumen therethrough, the inner sheath adapted to be rotatable and translatable within the central lumen of the catheter and to selectively cover or uncover at least one of the at least one side ports.

15. The system of claim 14, wherein the inner sheath further comprises at least one aperture through the elongate body of the inner sheath, the inner sheath configured to align the at least one aperture of the inner sheath with at least one side port.

16. The system of claim 15, further comprising a fluid infused through the central lumen of the inner sheath.

17. The system of claim 14. further comprising an inflatable balloon translatably disposed within the central lumen of the inner sheath.

18. A system comprising:
a catheter comprising an elongate body and a central lumen, and at least one side port defined through the elongate body; and
a side port control device embedded in or along the elongate body proximate the at least one side port.

19. The system of claim 18, further comprising a push/pull wire in operative connection with the side port control device, the push/pull wire extending proximally through the central lumen and adapted to actuate the side port control device to open or close the at least one side port.

20. The system of claim 19, wherein side port control device comprises a flap connected with the push/pull wire.

21. The system of claim 18, further comprising an external power source and electrical wires connected with the external power source and with the side port control device, wherein the side port control device is configured to cover or uncover a side port in response to an electrical signal sent from the external power source through the electrical wires.

22. The system of claim 21, wherein the side port control device comprises a shape memory material responsive to the electrical signals to cover or uncover the side port.

23. A system comprising:
a catheter comprising an elongate body and a central lumen, and at least one side port defined through the elongate body; and
a filter covering the at least one side port.

24. The system of claim 23, wherein the filter is adapted to only allow fluids of defined viscosity and/or molecules of a defined maximum size to pass through the filter.

25. A method for treating and/or preventing a condition within the vasculature of a patient, comprising:
providing a system according to claim 12;
advancing the catheter to a predetermined position within the vasculature, wherein at least one of the at least one side ports is located at a location within the vasculature for treating the condition;
infusing at least one therapeutic agent through the catheter lumen and through the at least one side port, wherein the at least one therapeutic agent contacts the vasculature; and
preventing and/or treating the condition within the patient's vasculature.

26. The method of claim 25, wherein the condition comprises vasospasm and the at least one therapeutic agent comprises an anti-vasospasm therapeutic agent.

27. The method of claim 25, wherein the condition comprises restenosis and the at least one therapeutic agent comprises an anti-restenosis agent.
